(19) United States
(12) Patent Application Publication
(10) Pub. No.: US 2007/0280930 A1
(43) Pub. Date: Dec. 6, 2007

(54) Cd44-Targeting for Reducing/Preventing Ischemia-Reperfusion-Injury

(76) Inventors: Kasper Mathias Antoon Rouschop, Leiden (NL); Sandrine Florquin, Oegstgeest (NL)

Correspondence Address: MORGAN LEWIS & BOCKIUS LLP, 1111 PENNSYLVANIA AVENUE NW, WASHINGTON, DC, 20004, US

(21) Appl. No.: 10/593,296

(22) PCT Filed: Mar. 16, 2005
(86) PCT No.: PCT/NL05/00200

§ 371 Date: Jul. 12, 2007

(30) Foreign Application Priority Data
Mar. 17, 2004 (EP) 04075867.4

(51) Int. Cl. A61K 39/395; A61K 31/715; A61P 37/06; G01N 33/50

(52) U.S. Cl. 424/133.1; 424/130.1; 436/87; 514/54; 514/789

(57) ABSTRACT

The present invention relates to CD44 binding molecules, preferably anti-CD44 antibodies, and their use in methods for the prevention or reduction of ischemia-reperfusion injury in e.g. solid organ transplantation or in patients in shock. The invention further relates to methods wherein levels of soluble CD44 are determined in e.g. serum or urine as a prognostic factor for the risk of organ rejection.

### FIELD OF THE INVENTION

The present invention relates to CD44 binding molecules, preferably anti-CD44 antibodies, and their use in methods for the prevention or reduction of ischemia-reperfusion injury in e.g. solid organ transplantation or in patients in shock. The invention further relates to methods wherein levels of soluble CD44 are determined in e.g. serum or urine as a prognostic factor for the risk of organ rejection.

### BACKGROUND OF THE INVENTION

Organ injury due to ischemia followed by reperfusion is a major clinical problem and is the most common cause of acute organ failure after transplantation, shock, sepsis and e.g. renal artery stenosis. Moreover, ischemia reperfusion injury is associated with a mortality rate of approximately 50%. Ischemia-reperfusion injury is characterized by the massive influx of neutrophils that exert a crucial role in the pathophysiology of post-ischemic failure of organs like kidneys by the release of cytotoxic proteases and oxygen derived radicals. Migration of neutrophils to the post-ischemic tissue is regulated by complement deposition such as C50α and a number of chemokines such as IL-8, Gro-α (murine homologue is cytokine-induced neutrophil chemoattractant (KC)) and macrophage inflammatory protein-2 (MIP-2). Yet adhesion, extravasation and infiltration of neutrophils into the post-ischemic tissue are at the basis of the development of ischemia-reperfusion injury. Therefore, we propose that the massive influx of neutrophils and the subsequent damage to the reperfused renal tissue may be the result, at least in part, from CD44-mediated adhesion and extravasation.

CD44 is a family of type I transmembrane glycoproteins with a wide tissue distribution including expression on leukocytes, epithelial and endothelial cells. CD44 family glycoproteins are encoded by single gene consisting of 19 exons. By alternative splicing, many different isoforms can be generated (Screaton et al., PNAS 1992, 89:12160-4; Günthert, Curr. Top Microbiol Immunol 1993 184:47-63; Tölg et al, NAR 1993 21:1225-9). These isoforms have been implicated in many physiological and pathological processes, such as cell-cell and cell-matrix interaction, leukocyte extravasation, wound-healing/scarring, cell migration, lymphocyte activation, and binding/presentation of growth factors (Weiss, L., S. Slavin, et al. (2000). Proc Natl Acad Sci USA 97(1): 285-90; Jones, M., L. Tussey, et al. (2000) J Biol Chem 275(11): 7964-74; Siegelman et al., J. Leukoc. Biol., 1999, 66:315-21; van der Voort et al., J. Biol. Chem. 1999, 274:6499-6506). Yet, all isoforms contain a hyaluronic acid (HA) and osteopontin binding site, which are the major ligands of CD44 (Siegelman et al., J. Leukoc. Biol., 1999; Weber et al., Science 1996, 271:509-12).

Little is known about the role of CD44 in the kidney after renal injury. Under normal conditions, CD44 is hardly expressed in the kidney except in passenger leukocytes. In inflammatory renal diseases, CD44 expression is markedly enhanced in crescents, on injured renal tubular epithelial cells (TECs) and capillary endothelial cells, as documented in both human diseases and in several animal models (see e.g. Florquin and Rouschop, Kidney Int. 2003, 86:S15-20). Specifically, after ischemia reperfusion injury CD44 expression is observed within 1 day after injury (Lewington, Am J Physiol Regul Integr Comp Physiol. 2000, 278:R247-54). The major source of CD44 found in the injured kidney is however derived from leukocytes and capillary endothelial cells. Knoflach et al. (J. Am. Soc. Nephrol. 1999, 10:1059-66) reported that low-molecular weight hyaluronate, which blocks the interactions between T lymphocyte CD44 and hyaluronate that is present on the surface of endothelial cells, is capable delaying acute rejection in a rat renal allograft model, however, only in combination with the immunosuppressant cyclosporine A. In organs other than kidney, Fujisaka et al. (J. Hep. Bil. Pancr. Surg. 1998, 5:196-99) reported strong expression of CD44 in portal areas of liver rejected after transplantation and Zander et al., (J. Heart Lung Transpl. 1999, 18:646-53) reported increased CD44 expression on graft-infiltrating inflammatory cells and resident parenchymal cells in lung allografts.

U.S. Pat. No. 6,001,356 disclose a method for preventing tissue destruction associated with autoimmune inflammatory diseases by utilizing anti-CD44 monoclonal antibodies to induce the loss of the CD44 receptor from cell surfaces, thus preventing the interaction between cell-surface CD44 and extracellular hyaluronan.

U.S. Pat. No. 6,506,382 discloses a method for inhibiting reperfusion injury using antibodies to P-selectin glycoprotein ligand.

It is an object of the present invention to provide 1) new therapeutic strategies based on CD44 to prevent or reduce organ tissue injury following ischemia/reperfusion injury following ie transplantation, shock, hypoperfusion, artery and 2) new diagnostic tools based on the measurement of soluble CD44 as a prognostic/diagnostic marker for rejection.

### DESCRIPTION OF THE INVENTION

The present invention relates to methods for preventing or reducing ischemia-reperfusion injury by administration of a CD44 blocking molecule. Injury to tissues, organs or groups of organs due to ischemia followed by reperfusion is a common cause of acute organ failure after transplantation, shock, sepsis, trauma and artery stenosis. More particularly, the invention provides a method of using a CD44 blocking molecule for prevention or reduction of ischemia-reperfusion injury in a solid organ. The CD44 blocking molecule is preferably administered in an amount effective in preventing or reducing ischemia-reperfusion injury as indicated below. Organs in which ischemia-reperfusion injury may be prevented or reduced in the methods of the invention include organs selected from the group consisting of kidney, liver, lungs, heart, small intestine and pancreas.

In a preferred method of the invention the ischemia-reperfusion injury is prevented or reduced in a subject undergoing solid organ transplantation by administration of an effective amount of (a composition comprising) a CD44 blocking molecule. The organ may be an organ as indicated above and may be a complete organ or part thereof. Preferably, in the method the CD44 blocking molecule or suitable compositions comprising the molecule is administered prior to transplantation of the organ. A composition comprising the CD44 blocking molecule may e.g. be administered to the subject undergoing transplantation in one or multiple intravenous injections. Alternatively or additionally, the CD44 blocking molecule is administered (ex vivo) to the solid organ (to be transplanted) by perfusion of the organ with a perfusion fluid comprising the CD44 blocking molecule. Preferably, the organ to be transplanted is perfused with the fluid comprising the CD44 blocking molecule shortly after removal of the organ from the donor or before transport or storage of the organ. This will improve preservation of the organ and diminish post transplantation ischemia-reperfusion injury.

In another preferred method, the ischemia-reperfusion injury is prevented or reduced in one or more solid organs in a subject in shock or septic shock by administration of an effective amount of (a composition comprising) a CD44 blocking molecule, e.g. in one or multiple intravenous injections. Particularly, the organ in which the ischemia-reperfusion injury is prevented or reduced in a subject in shock, includes the kidney. More particularly, the method is a method for the prevention or reduction of tubulus necrose.

In a further preferred method of the invention the ischemia-reperfusion injury is prevented or reduced in one or more organs, limbs, extremities or body parts that have been severed from the body and that are being re-attached to the body by reconstructive microsurgery. These method may comprise administration of an effective amount of (a composition comprising) a CD44 blocking molecule, e.g. in one or multiple intravenous injections to the patient, as well as, perfusion of the severed organs, limbs extremities or body parts prior to reconstruction.

For jurisdictions outside the USA, it is understood that the invention relates to the use of a CD44 blocking molecule in the manufacture of a medicament that may be applied in the methods of the invention.

In the methods and uses of the invention, the CD44 blocking molecule may be any molecule capable of binding and blocking CD44 in a manner that prevents or reduces ischemia-reperfusion injury. The CD44 blocking molecule preferably specifically binds to CD44. The term “specific binding,” as used herein, includes both low and high affinity specific binding. Specific binding can be exhibited, e.g., by a low affinity antibody or antibody-fragment having a Kd of at least about 10⁻⁴ M. Specific binding also can be exhibited by a high affinity antibody or antibody-fragment, for example, an antibody or antibody-fragment having a Kd of at least about of 10⁻⁷ M, at least about 10⁻⁸ M, at least about 10⁻⁹ M, at least about 10⁻¹⁰ M, or can have a Kd of at least about 10⁻¹¹ M or 10⁻¹² M or greater.

Whether a given compound is capable of binding and blocking CD44 in a manner that prevents or reduces ischemia-reperfusion injury may be determined in the mouse model assay for renal ischemia-reperfusion injury as described in the Examples herein. In this assay, a compound is defined to reduce renal ischemia-reperfusion injury as measured by renal function if, after 24 hours of reperfusion, serum creatinine or blood urea nitrogen (BUN) levels have not increased more than 90% of the increase of the corresponding serum level in control mice not treated with the compound.

Suitable CD44 blocking molecules can include antibody molecules, as well as homologues, analogues and modified or derived forms thereof, including immunoglobulin fragments like Fab, (Fab′)₂ and Fv, as well as small molecules including hyaluronate of low-, medium-, or high-molecular weight, peptides, oligonucleotides, peptidomimetics and organic compounds which bind to and block CD44 as defined above.

The term “antibody” refers to a member of a family of glycosylated proteins called immunoglobulins, which can specifically combine with an antigen. The term as used herein is intended to include all classes of immunoglobulins (IgG, IgM, IgA, IgD, or IgE), poly- and monoclonal antibodies, single chain antibodies, antigen binding fragments (e.g., Fab, F(ab′)₂, Fab′) as well as whole immunoglobulins. The term “monoclonal antibody” (mAb) means an antibody population having a homogenous antibody composition, each number of which binds to the same antigenic determinant(s).

The antibody for use in the present invention is an antibody that binds to and blocks CD44 as defined above. The antibody is preferably capable of binding and blocking the constant form of CD44. One such preferred antibody is an antibody, which has the same recognition site on CD44 as the IM7 antibody as described in U.S. Pat. No. 6,001,356, which incorporated herein by reference. Such a preferred antibody is thus capable of cross-blocking the IM7 antibody whereby an antibody is defined to cross-block the IM7 antibody if it is capable of reducing the binding of IM7 to CD44 by at least 10%. The mAb IM7 was generated using a myeloid cell line from mouse bone marrow for the immunisation of rats (Trowbridge et al., Immunogenetics, 15 (1982) 299-312). The hybridoma is commercially available from American Type Culture Collection as “Rat hybridoma, clone IM7.8.1, producing anti-mouse Pgp-1 mAB”. Although mAb IM7 was raised against murine CD44, IM7 also recognizes human CD44 and binds to an epitope in the non-variable region of both human and mouse CD44 (Peach et al., J. Cell Biol., 122 (1993) 257-264). A 13-amino acid-long sequence (NH2-Asp-Leu-Pro-Asn-Ser-Phe-Asp-Gly-Pro-Val-Thr-Ile-Thr-COOH) between residues 115 and 127 of murine CD44 comprises the IM7 epitope.

U.S. Pat. No. 6,001,356 discloses the amino acid sequence of the binding site of antibody IM7 on CD44 and that a synthetic CD44 peptide containing the binding site of IM7 is able to block the antibody in vitro and in vivo. Hence, should any imbalance (i.e. overdose, unexpected side effects, allergy) occur during the administration of an antibody containing the same recognition site on CD44 as the original IM7 antibody IM7 recognition site, the synthetic CD44 peptide can be utilized as an antidote to quickly neutralize the antibody and provide control over the effects of the antibody.

Other (monoclonal) antibodies with the required specificity may be generated by methods that are well-known to the skilled person (see e.g. “Using Antibodies”, E. Harlow and D. Lane, Cold Spring Harbor Laboratory Press, 1999).

For treating humans, the anti-CD44 antibodies or mAbs would preferably be used as chimeric, deimmunised (Deimmunised™), humanised or human antibodies. Such antibodies can reduce immunogenicity and thus avoid human anti-mouse antibody (HAMA) response. Optionally the antibody be IgG4, IgG2, or other genetically modified IgG or IgM that do not augment antibody-dependent cellular cytotoxicity (S. M. Canfield and S. L. Morrison, J. Exp. Med., 1991: 173: 1483-1491) and complement mediated cytolysis (Y. Xu et al., J. Biol. Chem., 1994: 269: 3468-3474; V. L. Pulito et al., J. Immunol., 1996; 156: 2840-2850).

Chimeric antibodies are produced by recombinant processes well known in the art, and have an animal variable region and a human constant region. Humanized antibodies have a greater degree of human peptide sequences than do chimeric antibodies. In a humanized antibody, only the complementarity determining regions (CDRs) which are responsible for antigen binding and specificity are animal derived and have an amino acid sequence corresponding to the animal antibody, and substantially all of the remaining portions of the molecule (except, in some cases, small portions of the framework regions within the variable region) are human derived and correspond in amino acid sequence to a human antibody. See L. Riechmann et al., Nature, 1988; 332: 323-327; G. Winter, U.S. Pat. No. 5,225,539; C. Queen et al., U.S. Pat. No. 5,530,101.

Deimmunised antibodies are antibodies in which the T and B cell epitopes have been eliminated, as described in International Patent Application PCT/GB98/01473. They have reduced immunogenicity when applied in vivo.

Human antibodies can be made by several different ways, including by use of human immunoglobulin expression libraries (Stratagene Corp., La Jolla, Calif.) to produce fragments of human antibodies (VH, VL, Fv, Fd, Fab, or (Fab′)₂, and using these fragments to construct whole human antibodies using techniques similar to those for producing chimeric antibodies. Human antibodies can also be produced in transgenic mice with a human immunoglobulin genome. Such mice are available from Abgenix, Inc., Fremont, Calif., and Medarex, Inc., Annandale, N.J.

One can also create single peptide chain binding molecules in which the heavy and light chain Fv regions are connected. Single chain antibodies (“ScFv”) and the method of their construction are described in U.S. Pat. No. 4,946,778. Alternatively, Fab can be constructed and expressed by similar means (M. J. Evans et al., J. Immunol. Meth., 1995; 184: 123-138).

All of the wholly and partially human antibodies are less immunogenic than wholly murine mAbs, and the fragments and single chain antibodies are also less immunogenic. All of these types of antibodies are therefore less likely to evoke an immune or allergic response. Consequently, they are better suited for in vivo administration in humans than wholly animal antibodies, especially when repeated or long-term administration is necessary. In addition, the smaller size of the antibody fragment may help improve tissue bioavailability, which may be critical for better dose accumulation in acute disease indications as in the methods of the present invention.

Based on the molecular structures of the variable regions of the anti-CD44 mAbs one could use molecular modeling and rational molecular design to generate and screen molecules which mimic the molecular structures of the binding region of the antibodies or the peptides, respectively, and which prevent or reduce ischemia-reperfusion injury as defined above. These small molecules can be peptides, peptidomimetics, oligonucleotides, or other organic compounds. Alternatively, one could use large-scale screening procedures commonly used in the field to isolate suitable molecules from libraries of compounds.

The CD44 blocking molecule may be formulated with conventional pharmaceutically acceptable parenteral vehicles for administration by injection. Such vehicles are inherently non-toxic and non-therapeutic. Examples are water, saline, Ringer's solution, dextrose solution, and Hank's solution. The formulation may contain minor amounts of additives such as substances that maintain isotonicity, physiological pH (e.g., buffers) and stability (preservatives). The blocking molecule is prepared in purified form substantially free of other proteins, endotoxins and other contaminants, and stored as a sterile, preferably lyophilized (freeze-dried) powder. The solution of blocking molecules, free of aggregates, is formulated in sterile isotonic liquid at concentrations of about 1 to about 10 mg per ml and administered intravenously to patients during a period of several hours. Slow administration permits continuous monitoring of the vital functions of the patient. Experimental animals are also treated parenterally; intravenous administration can be used for larger animals and smaller ones can be injected intraperitoneally.

The blocking molecules used in the method of the present invention are preferably administered to individuals, preferably mammals, more preferably humans, in a manner that will maximize the desired effect. The blocking molecule or antibody may be administered prior to, or at the onset of, or during reperfusion.

The dose for individuals of different species and for different diseases is determined by measuring the effect of the blocking molecule or antibody on the lessening of those parameters, which are indicative of the disease being treated. In case the blocking molecule is an antibody it will normally be administered parenterally, typically intravenously, as a bolus or in an intermittent or continuous regimen. The dose will depend upon the patient and the patient's medical history.

Suitable pharmaceutical vehicles and their formulations are described in “Remington's Pharmaceutical Sciences” by E. W. Martin which is incorporated herein by reference.

In mouse models of ischemia-reperfusion injury, a single intraperitoneal or intravenous injection of 100 μg of IM7 antibody produced measurable reduction of renal injury as measured by renal function (FIG. 2). U.S. Pat. No. 6,001,356 reports that antibody IM7 has the same effect on human cells in vitro as for the mouse system. Hence, dosage rates for humans can be extrapolated based on the results of animal data. For human use, for example in patients undergoing an organ transplantation, the patient may receive one or multiple intravenous injections of CD blocking molecules or anti-CD44 antibodies before transplantation to prevent ischemia-reperfusion injury in a dosage ranging from about 1 to about 15 mg per kg body weight, preferably from about 2 to about 8 mg per kg body weight, more preferably from 3 to about 6 mg per kg body weight and most preferably about 4 mg per kg body weight. Effective treatment is reflected by clinical assessment and laboratory measurements (e.g., renal function as measured by serum markers. The method of administering the dosage may be varied by the treating physician due to patient condition and the severity of the condition being treated.

For perfusion of (donor) organs before transplantation to remove donor blood, to increase the preservation of the organ and to diminish ischemia-reperfusion injury organs are “rinsed” by a suitable perfusion fluid comprising the CD44 blocking molecule such as an antibody. The CD44 blocking molecule or anti-CD44 antibodies is added to the perfusion fluid in a dose of about 1 to about 15 μg per g of organ, preferably from about 2 to about 8 μg per g of organ, more preferably from 3 to about 6 μg per g of organ and most preferably about 4 μg per g of organ.

In a further aspect, the invention relates to a method for prognoses of the risk of rejection of a transplanted organ, wherein the method comprises the step of measuring the level of soluble CD44. Preferably, the level of soluble CD44 is measured prior to transplantation of the organ. The level is measured in a biological sample and preferably, this is done ex vivo. Biological samples in which soluble CD44 are determined may include e.g. blood, a blood fraction (such as e.g. serum), urine or a urine fraction. The method of prognosis may be applied to subject into whom an organ is to be transplanted that is selected from the group consisting of kidney, liver, lungs, heart, small intestine and pancreas or onto whom a severed organ, limb extremity or body part is reattached. In the method, a serum CD 44 level in excess of 600, 700, 800, 900 or 1000 ng of soluble CD44 per ml serum may be taken as indicative for a high risk of organ rejection.

The level of soluble CD44 may be determined in a variety of ways known to the skilled person. Preferably, an anti-CD44 antibody as described herein is used in an immunoassay, preferably a quantitative immunoassay. A large variety of immunoassays are available in the art including e.g. ELISA's (as exemplified in the Examples herein), blotting techniques such as Western blotting, and RIAs, etc., for which reference is made to the standard handbooks (see e.g. Harlow and Lane, 1988, “Antibodies: A Laboratory Manual”, Cold Spring Harbor Laboratory, Cold Spring Harbor Laboratory Press, New York) as well as for instance WO 93/18152.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

### DESCRIPTION OF THE FIGURES

FIG. 1 Post-reperfusion serum levels of creatinine (A) and ureum (B) in CD44+/+ and CD44−/− mice.

FIG. 2 Post-reperfusion serum levels of creatinine and ureum in control mice (CD44+/+) and in mice treated with anti-CD44 antibody.

FIG. 3 Pre-transplantation serum levels of soluble CD44 measured by ELISA in patients undergoing kidney transplantation and in healthy controls.

### EXAMPLES

### Example 1

### Deficiency or Blocking of CD44 Reduces Ischemia Reperfusion Injury

1.1 Materials and Methods

1.1.1 Mice and Experimental Protocol

Bilateral ischemia or sham surgery was performed under general anesthesia (0.07 ml/10 g mouse of FFM mixture, containing: 1.25 mg/ml midazolam (Roche, Mijdrecht, The Netherlands), 0.08 mg/ml fentanyl citrate and 2.5 mg/ml fluanisone (Janssen Pharmaceutica, Beerse, Belgium)) in 6-8 weeks old, male mice. Mice, CD44 knock-out on C57B1/6 background (CD44−/−) (Schmits R, F. J. et al, Blood, 1997, 90: 2217-33) and C57B1/6 wild type (CD44+/+) origin were bred in our animal facility. Antibody treated mice received 16 hours prior to surgery a single intra-peritoneal injection of 100 μg anti-CD44 (clone IM7, rat IgG_{2b}, ATCC, Livermore, Calif.) or control IgG (purified rat IgG2b, Pharmingen, Erembodegem, Belgium) (Brennan et al., 1999 Immunology 98(3): 427-35; Weiss et al., 2000, Proc Natl Acad Sci USA 97(1): 285-90). Renal arteries and veins were bilaterally occluded for 45 minutes with microaneurysm clamps, during which the mice were placed in a 32° C. stove. All mice received postoperative analgesia (0.15 mg/kg buprenorfine, subcutaneously (Shering-Plough, Brussel, Belgium)). Sham operated mice underwent the same procedure without clamping of the arteries and veins. To mark proliferating cells, 5-bromo-2′-deoxyuridine (BrdU; Sigma chemical Co., St. Louis, Mo.) was injected intraperitoneally (50 mg/kg body weight) one hour prior to sacrifice. Groups of mice were sacrificed 1, 3, 7 and 14 days after surgery by exsanguination under general anesthesia. All experimental procedures were approved by the Animal Care and Use Committee of the University of Amsterdam, the Netherlands.

1.1.2 Antibodies and Chemicals

Rat IgG2b anti-CD44 was obtained from the concentrated supernatant of the hybrydoma IM 7.8.1 (ATCC, Livermore, Calif.) and purified by protein G-sepharose chromatography (Calbiochem, Darmstadt, Germany). The IM7 anti-CD44 antibody is described in U.S. Pat. No. 6,001,356. Goat anti-osteopontin was purchased from R&D systems (Abingdon, UK). The biotinylated HA binding protein was obtained from Calbiochem. BrdU and the antibody against BrdU were purchased from Sigma. Apoptosis was determined by the use of an antibody directed to cleaved caspase 3 (Cell signalling Technology, Beverly, Mass.). Macrophages were detected with rat anti-F4/80 (Serotec, Oxford, UK). Granulocytes were detected by Ly6-G (Pharmingen, Erembodegem, Belgium). HRP-labeled secondary antibodies were obtained from DAKO (Glostrup, Denmark).

1.1.3 Histology Immunohistochemistry and Renal Function

Renal tissues were fixed in 10% formalin for 12 hours and embedded in paraffin in a routine fashion. Sections were cut, deparaffinized and stained with hematoxylin and eosin (HE) or periodic acid schiff (PAS). For detection of CD44, osteopontin, macrophages, granulocytes and apoptosis, antigen retrieval was performed by microwave treatment. To detect BrdU, DNA was denatured in 2N HCl and antigen retrieval was performed by 0.4% pepsin (Sigma chemicals). After blocking of endogenous peroxidase activity with H₂O₂, free protein binding sites were blocked with normal goat serum, except for the sections afterwards probed with goat anti-osteopontin (blocked with human serum) and biotinylated HA binding protein (blocked with bovine serum albumin). After blocking, the sections were probed with the antibody. As a negative control we used species and isotype matched antibodies. After incubation with the secondary (HRP-labeled) antibody, bound antibodies were visualized by developing peroxidase activity using 3,3-diamino-benzidine tetrachloride (DAB, Sigma). The slides were counterstained with methyl green (Sigma).

Blood urea nitrogen (BUN) was determined using a urease assay. Serum creatinine was determined using a standard creatininase assay.

1.1.4 Preparation and Administration of Labeled Murine Neutrophils (PMNs)

Homozygous C57B1/6J-CD44 knock-out and wild type C57B1/6J mice were anesthetized, and 0.5 to 1.0 mL of blood was withdrawn from each mouse by percutaneous intracardiac puncture with a 22-gauge needle and transferred to a sterile test tube containing heparin at room temperature. Blood was then diluted 1:1 with PBS, transferred to a 15-mL conical tube containing an equal volume Ficoll-Hypaque (Pharmacia LKB Technology), and centrifuged at 1800 rpm for 20 minutes at room temperature. The buffy coat was then gently transferred to a second conical tube and centrifuged at 1400 rpm for 15 minutes at 4° C. The supernatant was aspirated, and red blood cells were subjected to hypotonic lysis; the remaining cells were then resuspended in PBS. The sample was centrifuged at 1200 rpm for 12 minutes at 4° C., the supernatant was decanted, and the hypotonic lysis step was repeated until the specimen was free of erythrocytes.

The leukocytes were then resuspended in PBS to a count of 5×106 cells/mm³ and incubated at 37° C. with 0.5 or 1 μM Celltracker Green (C57B1/6) or Celltracker Orange (CD44−/−) (Molecular probes, Leiden, the Netherlands) in medium, after for 15-45 minutes 10% NMS was added. The neutrophils were then centrifuged at 1800 rpm for 5 minutes and washed 3 times with medium containing 10% NMS at 37° C. The WT and CD44−/− neutrophils were then resuspended to a final concentration of 2.0×106 cells/mL. (1*10e6 WT+1*10e6 KO). This mixture was injected intravenously to animals 1 hour prior to surgery.

1.1.5 Elisa's

The Elisa used for detection of soluble CD44 in serum was purchased from Bender Medsystems (Vienna, Austria) and was performed according to the manufactures instruction.

1.1.6 Histopathological Scoring

Tubular injury, characterized by necrosis, dilatation, cast deposition and loss of brush border were graded to the extent of outer medulla involvement in 10 randomly chosen, non-overlapping fields (×200 magnification), on a scale from 0 to 5:

0=normal; 1=very mild, involvement of less than 10% of the outer medulla; 2=mild, involvement of 10-25% of the outer medulla; 3=moderate, involvement of 25-50% of the outer medulla; 4=severe damage, involvement of 50-75% of the outer medulla; 5=extensive damage involving more than 75% of the outer medulla. These values were expressed as tubular injury scores. Granulocytes and macrophages were counted in a random non-overlapping total of 10 fields (×200 magnification), data are expressed per mm². To evaluate proliferation and apoptosis of TECs, the number of respectively BrdU and active caspase-3 positive apoptotic tubular cells were counted in 10 non-overlapping fields (×200), and expressed as apoptotic cells per mm².

Osteopontin was expressed as the percentage of positive tubuli. An area of 5 mm² was analyzed for hyaluronic acid using a digital image analysis program (Image pro-plus®, Mediacybernetics, Germany), values are expressed as a percentage of the total cortex.

1.2 Results

1.2.1 Renal CD44 After Ischemia Reperfusion Injury

In normal mice, after bilateral renal pedicle clamping and reperfusion CD44 expression was increased when compared to sham-operated animals. After 1 day of reperfusion CD44-expression was detected on peritubulary endothelium but was most pronounced at infiltrating cells in the interstitium. After 3, 7 and 14 days of reperfusion CD44-expression increased further and, besides leukocytes and endothelial cells, was also detected laterally on tubular epithelial cells in the outer medulla. To study the role of CD44 in the development of renal ischemia-reperfusion injury, we subjected CD44 knock-out mice (CD44^{-/-}) to ischemia reperfusion and comparison was made to wild-type mice (CD44^{+/+}).

1.2.2 CD44-Deficiency Preserved Renal Function and Decreased Renal Injury

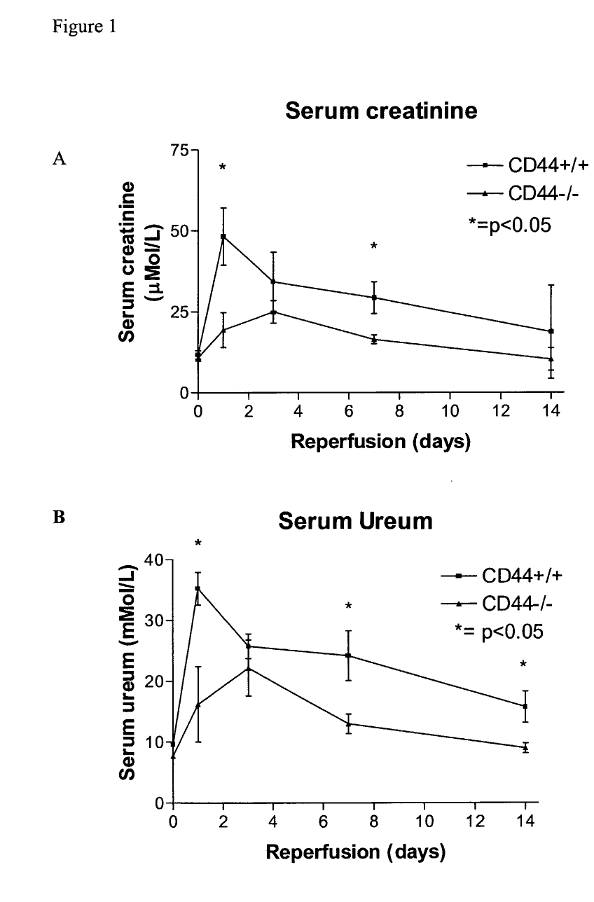

After 24 hours of reperfusion, serum creatinine and BUN levels increased more than 4-fold over baseline in CD44^{+/+} mice. In contrast, in CD44^{-/-} creatinine and ureum increased less than 2-fold (FIGS. 1A and B). In accordance, histological changes in the outer medulla concerning tubular damage were attenuated in CD44−/− mice.

1.2.3 Hyaluronic Acid and Osteopontin

Since HA and osteopontin are the principal ligands of CD44 and promote inflammation, we analyzed HA and osteopontin expression by immunohistochemistry. Interstitial HA-positive areas expanded in the post-ischemic kidneys of both genotypes. Interestingly, resolution of HA was severely hampered in the absence of CD44. In contract, osteopontin expression was comparable in CD44^{-/-} and CD44^{+/+} obstructed kidneys.

1.2.4 CD44-Deficiency Diminished Granulocyte and Macrophage Infiltration

Histologic examination indicated that CD44 regulates, at least in part, the influx of granulocytes into the post-ischemic kidney, 1 day after ischemia was induced. In contrast, the number of granulocytes 3 and 7 days after induction of ischemia are increased in CD44^{-/-} compared to CD44^{+/+}.

Influx of macrophages into the post-ischemic kidney was decreased at all time-points in CD44^{-/-} compared to CD44^{+/+} after the induction of ischemia.

To determine whether the difference in influx of granulocytes was mediated by the production of chemotactic factors by the post-ischemic kidney, renal lysates were assessed for KC and MIP-2. Although both MIP-2 and KC were elevated at 1 day and 3 days after ischemia, no differences between CD44^{+/+} and CD44^{-/-} were detected that could explain the striking difference in granulocyte influx.

1.2.5 Anti-CD44 Reduces Ischemia Reperfusion Injury in Mice

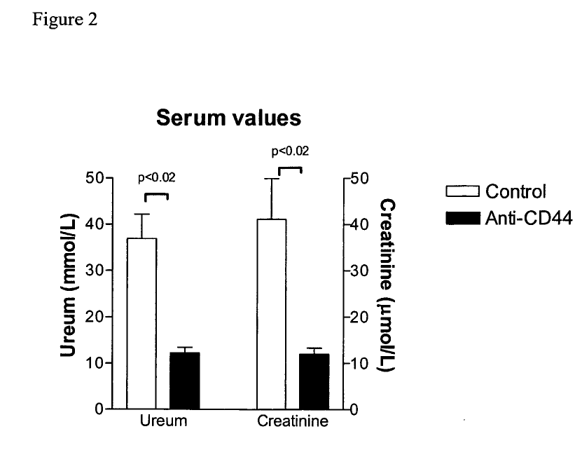

To exclude compensatory mechanisms in the CD44^{-/-} mice and to study the therapeutic potential CD44-blockade, an anti-CD44 antibody (IM7, see above) was used to block CD44 in a second independent experiment. Antibody was administered as indicated above. Administration of a blocking anti-CD44 antibody resulted in a decreased neutrophil influx into the post-ischemic kidney and preservation of the renal function after ischemia-reperfusion injury (FIG. 2). CD44 is therefore useful as a novel therapeutic target in controlling renal ischemia reperfusion injury since deficiency or blocking CD44 resulted in a decreased influx of neutrophils and subsequently preservation of the renal function following ischemia-reperfusion of the post-ischemic kidney.

### Example 2

### Prognostic Value of Serum CD44 Levels for Renal Allograft Rejection

2.1 Patients

Patients were randomly selected from the patient population of the Academic Medical Center of the University of Amsterdam undergoing renal transplantation. Urine and serum samples from all patients were collected 24 hours before renal transplantation was performed. at the time of biopsy. The patients were followed for more than one year and clinical episodes of rejection assessed and confirmed by renal biopsies. There were no statistical difference between the rejecting and non-rejecting patients regarding sex, age, renal function before transplantation, immunosuppressive treatment, primary renal disease. Serum samples were also obtained from a control group of 10 non-transplanted healthy volunteers

2.2 ELISA's

The Elisa used for detection of soluble CD44 in serum was purchased from Bender Medsystems (Vienna, Austria) and was performed according to the manufactures instruction.

2.3 Results

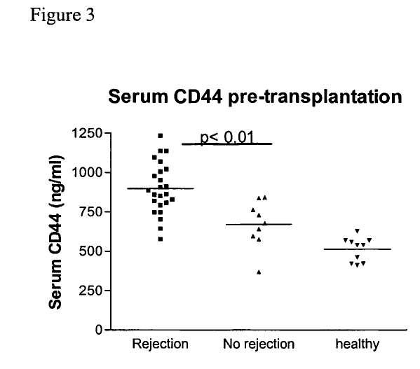

Serum levels of soluble CD44 were measured in patients undergoing renal transplantation 24 hours prior to transplantation, using the ELISA assay described above. Using the same procedure CD44 serum levels in healthy control subject were determined. The results are summarised FIG. 3 and indicate that pre-transplantation serum levels of soluble CD44 correlate with rejection of the transplanted kidney. Levels in excess of 600 ng CD44 per ml serum are prognostic for rejection of the transplanted organ.

1-13. (canceled)

14. A method for prognoses of the risk of rejection of a transplanted organ, reconstructed organ, limb, extremity or body part, wherein the method comprises the step of measuring the level of soluble CD44.

15. (canceled)

16. A method according to claims 14, wherein the level of soluble CD44 is measured prior to transplantation or reconstruction of the organ, limb, extremity or body part.

17. A method according to any one of claims 14, wherein the level of soluble CD44 is measured ex vivo in a biological sample.

18. A method according to any one of claims 14, wherein the biological sample is a blood, a blood fraction, serum, urine or a urine fraction.

19. A method according to any one of claims 14, wherein the organ is selected from the group consisting of kidney, liver, lungs, heart, small intestine and pancreas.

20. A method according to any one of claims 14, wherein a serum CD 44 level in excess of 600 ng soluble CD44 per ml serum is indicative for a high risk of rejection of the organ, limb, extremity or body part.

21. A method for prevention or reduction of ischemia-reperfusion injury in a mammal comprising administration of a CD44 blocking molecule.

22. The method of claim 21, wherein the CD44 blocking molecule is administered to a subject undergoing solid organ transplantation.

23. The method of claim 22, wherein the solid organ is selected from the group consisting of kidney, liver, lungs, heart, small intestine and pancreas.

24. The method of claim 22, wherein the CD44 blocking molecule is administered prior to transplantation.

25. The method of claim 24, wherein the CD44 blocking molecule is administered in one or more intravenous injections.

26. The method of claim 24, wherein the CD44 blocking molecule is administered by perfusion of the solid organ with a solution comprising the CD44 blocking molecule.

27. The method of claim 21, wherein the ischemia-reperfusion injury is prevented or reduced in one or more solid organs.

28. The method of claim 21, wherein the mammal is in shock.

29. The method of claim 27, wherein the solid organ is the kidney.

30. The method of claim 29, wherein the CD44 blocking molecule prevents or reduces tubulus necrose.

31. The method of claim 24, wherein the ischemia-reperfusion injury is prevented or reduced in one or more one or more organs, limbs, extremities or body parts that have been severed from the body and that are being re-attached to the body by reconstructive microsurgery.

32. The method of claim 21 wherein the CD44 blocking molecule is an anti-CD44 antibody or low-molecular weight hyaluronate.

33. The method of claim 31, wherein the anti-CD44 antibody is an antibody capable of cross-blocking the IM7 antibody by at least 10%.

34. The method of claims 31, wherein the antibody is a chimeric, deimmunised, humanized, or human antibody.
